(19) United States
(12) Patent Application Publication
(10) Pub. No.: US 2002/0020415 A1
(43) Pub. Date: Feb. 21, 2002

(54) Distributor for underwater breathing apparatus

(76) Inventor: Nino Pietrelli, Sori (IT)

Correspondence Address: LARSON & TAYLOR, PLC, 1199 NORTH FAIRFAX STREET, SUITE 900, ALEXANDRIA, VA, 22314, US

(21) Appl. No.: 09/768,239

(22) Filed: Jan. 25, 2001

(30) Foreign Application Priority Data
Aug. 18, 2000 (IT) GE2000U000029

(51) Int. Cl. A62B 7/00; A62B 9/00; A61M 15/00; A61M 16/00; A62B 18/00; A62B 9/02

(52) U.S. Cl. 128/205.24; 128/204.18

(57) ABSTRACT

Distributor for underwater breathing apparatus, comprising: a box-shaped body housing a device for regulating the air flow coming from a source of pressurized air; a tube connected to said box-shaped body and showing a mouthpiece; an air intake duct connected to said box-shaped body by means of an intake valve; and one or more discharge ducts connected to said box-shaped body. Within said distributor, on the lower edge of said tube showing the mouthpiece, barriers are provided against possible water seepages into the box-shaped body, said barriers being made so as to leave the passage section of said tube connected to the mouthpiece unchanged.

### BACKGROUND AND SUMMARY OF THE INVENTION

The present invention relates to distributors for underwater breathing apparatus.

As is known, distributors for underwater breathing apparatus comprise a box-shaped body housing a device for regulating the air flow coming from an intake duct connected by means of a suitable valve to a source of compressed air, such as diving bottles, and being sent to a tube showing a mouthpiece. Said air flow coming from the intake duct goes through a chamber provided in the regulation device and communicating with the tube showing the mouthpiece or, in case of a distributor provided with by-pass of said chamber, it goes through said by-pass and reaches directly said tube showing the mouthpiece.

In both types of distributor, with or without by-pass, some problems may occur linked with water seepage, above all from the air intake duct to the regulation device within the distributor body. Said water seepages, which have reached the chamber of said regulation device, may enter the mouthpiece connection tube as drops and probably reach the user's mouth making breathing seriously difficult.

The aim of the present invention is to overcome the disadvantages of known above-mentioned distributors, therefore providing for a distributor ensuring the user that possible water seepages into the box-shaped body, and in particular into the regulation device chamber, do not reach the mouthpiece.

Said aim is reached by the present invention by means of a distributor for underwater breathing apparatus, comprising a box-shaped body housing a device for regulating the air flow coming from a source of pressurized air; a tube connected to said box-shaped body and showing a mouthpiece; an air intake duct connected to said box-shaped body by means of an intake valve; and one or more discharge ducts connected to said box-shaped body; within said distributor, on the lower edge of said tube showing the mouthpiece, barriers are provided against possible water seepages into the box-shaped body, said barriers being made so as to leave the passage section of said tube connected to the mouthpiece unchanged.

According to further aspects of the present invention said barriers consist of a ring-shaped element connected to the lower edge of said tube and extended for a given portion within the regulation system chamber.

### BRIEF DESCRIPTION OF THE DRAWINGS

The following description will refer as a mere non limiting example to a distributor provided with by-pass known from patent application no. EP 0 937 640 A1 belonging to the owner of the present application. Said description will point out further aims and advantages of the present invention and will refer to the only enclosed drawing, in which:

FIG. 1 shows a top view partially sectioned of a distributor for underwater breathing apparatus according to the present invention.

### DESCRIPTION OF THE PREFERRED EMBODIMENT OF THE INVENTION

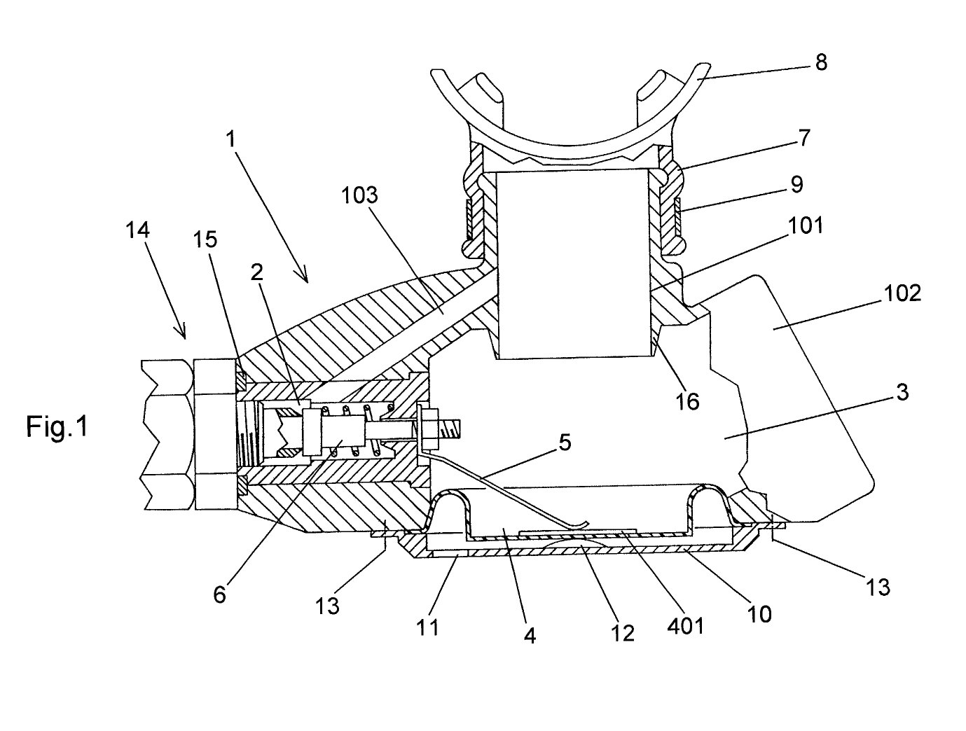

FIG. 1 shows a distributor for underwater breathing apparatus comprising a box-shaped body 1 housing a device (3, 4, 5, 6) for regulating the air flow coming from an intake duct 2. Said regulation device substantially comprises a chamber 3 having a flexible membrane 4 on the end of said box-shaped body 1 opposite to a mouthpiece 8; said flexible membrane 4, due to the pressure differential resulting when the scuba-diver inhales, operates the lever 5 coupled in a known way with the valve 6 for the regulation of the air flow, so that said valve 6 opens the intake duct 2. Said duct 2 is connected by means of a coupling 14 to a source of pressurized air, sealing means 15 being provided for said connection. The box-shaped body 1 further comprises an integral tube 101 coupling with a connection 7 of the mouthpiece 8, said connection 7 being advantageously made of an elastomeric material and provided with a sealing ring 9 for the distributor. The box-shaped body 1 further comprises a discharge duct 102, placed laterally with respect to the diver's mouthpiece 8, and a by-pass tube 103 obtained integrally within the box-shaped body 1, for instance by molding of the plastic material constituting said box-shaped body. As can be seen from the figure, the tube 103 takes air downstream from the regulation valve 6 and sends it directly to the tube 101 of the mouthpiece 8, thus by-passing the chamber 3 and creating within said chamber a depression which makes it easier for the stiff portion 401 of the flexible membrane 4 to act upon the lever 5. The distributor further comprises a stiff cap 10 on the flexible membrane 4. Said cap 10 is provided with an opening 11 for the communication of said membrane 4 with the surrounding water, so as to create between the surfaces of the latter the pressure differential which is necessary for the working of the regulation device. In this embodiment said cap 10 is fixed by means of pins 13 to the box-shaped body 1, and the flexible membrane 4 being introduced on its ends between the box-shaped body 1 and the cap 10 ensures an efficient sealing of the breathing apparatus. There is also, in a know way, a button 12 as bleeder and as distributor control.

As is evident, the air intake duct 2 is connected by means of the coupling 14 to a source of pressurized air. Although sealing means 15 are provided during inhalation, water seepages may occur, which enter the chamber 3 in the form of drops when the valve 6 is pushed towards the left by the lever 5, i.e. when the scuba diver breathes in. In order to avoid that these water drops reach the tube 101 of the mouthpiece 8, and therefore the diver's mouth, the lower edge of said tube 101 extends for a certain portion within the chamber 3, so as to form a ring-shaped element 16 thus constituting a barrier against which the water drops resulting from a possible seepage clash, thus avoiding to reach the mouthpiece 8. Said ring-shaped element 16 in the present embodiment is obtained integrally with the tube 101, though it could also be a separate element which can be connected to the lower edge of said tube 101.

I claim:

1. Distributor for underwater breathing apparatus, comprising a box-shaped body housing a device for regulating the air flow coming from a source of pressurized air; a tube connected to said box-shaped body and showing a mouthpiece; an air intake duct connected to said box-shaped body by means of an intake valve; and one or more discharge ducts connected to said box-shaped body, in which on the lower edge of said tube showing the mouthpiece barriers are provided against possible little water seepages into the box-shaped body, said barriers being made so as to leave the passage section of said tube connected to the mouthpiece unchanged.

2. Distributor according to claim 1, in which said barriers comprise at least a ring-shaped element connected to the lower edge of said tube and extending for a given portion within a chamber provided in the regulation device.

3. Distributor according to claim 2, in which said ring-shaped element is obtained integrally as an extension of the lower edge of the said tube showing the mouthpiece.
